(19) United States
(12) Patent Application Publication
(10) Pub. No.: US 2008/0161395 A1
(43) Pub. Date: Jul. 3, 2008

(54) Derivatives of Aminobutanoic Acid Inhibiting Cpt

(75) Inventors: Fabio Giannessi, Pomezia (IT); Emanuela Tassoni, Ciampino (IT); Maria Ornella Tinti, Rome (IT); Roberto Conti, Rome (IT); Natalina Dell'Uomo, Pomezia (IT); Tiziana Brunetti, Pomezia (IT)

Correspondence Address: LUCAS & MERCANTI, LLP, 475 PARK AVENUE SOUTH, 15TH FLOOR, NEW YORK, NY, 10016, US

(73) Assignee: SIGMA-TAU INDUSTRIE FARMACEUTICHE RIUNITE S.P.A., Rome (IT)

(21) Appl. No.: 11/815,068

(22) PCT Filed: Feb. 13, 2006
(86) PCT No.: PCT/EP06/01290

§ 371 Date: Sep. 24, 2007

(30) Foreign Application Priority Data
Mar. 2, 2005 (IT) RM2005A000090
Feb. 13, 2006 (EP) PCT/EP2006/001290

(51) Int. Cl. A61K 31/21; C07C 229/02; A61P 3/10

(52) U.S. Cl. 514/513; 560/169

(57) ABSTRACT

The invention relates to a new class of compounds with action inhibiting carnitine palmitoyl transferase (CPT), pharmaceutical compounds which contain at least one new compound according to the invention, and their therapeutic use in the treatment of hyperglycaemic conditions such as diabetes and the pathologies associated with it, congestive heart failure and obesity.

### FIELD OF THE INVENTION

The present invention describes a new class of compounds capable of inhibiting carnitine palmitoyl transferase (CPT); the invention also relates to pharmaceutical compositions, which comprise at least one new compound according to the invention, and their therapeutic use in the treatment of hyperglycaemic conditions such as diabetes and the pathologies associated with it, such as for example congestive heart failure and obesity.

### BACKGROUND OF THE INVENTION

Known hypoglycemic treatment is based on the use of drugs with a different mechanism of action (Arch. Intern. Med. 1997, 157, 1802-1817).

The more common treatment is based on insulin or its analogues, which uses the direct hypoglycemic action of this hormone.

Other compounds act indirectly by stimulating the release of insulin (sulfonylurea ureas). Another target of the hypoglycaemic drugs is the reduction of the intestinal absorption of glucose via the inhibition of the intestinal glucosidases, or the reduction of insulin resistance. Hyperglycaemia is also treated with inhibitors of gluconeogenesis such as the biguanides.

Some authors have shown the relationship between gluconeogenesis and the enzyme carnitine palmitoyl transferase.

Carnitine palmitoyl transferase catalyses the formation in the cytoplasm of palmitoyl carnitine (activated fatty acid) from carnitine and palmitoyl coenzyme A. Palmitoyl carnitine is different from palmitic acid in that it easily crosses the mitochondrial membrane. Palmitoyl coenzyme A reconstitutes itself within the mitochondrial matrix, releasing carnitine. Palmitoyl coenzyme A is oxidised to acetyl-coenzyme A, which activates pyruvic carboxylase, a key enzyme in the gluconeogenic pathway.

Some authors report that diabetic patients have high blood levels of fatty acids which are oxidised in the liver producing acetylcoenzyme A, ATP and NADH. The high availability of these substances causes over-regulation of gluconeogenesis, with a subsequent increase in the level of blood glucose. In these situations, the inhibition of CPT would limit the oxidation of the fatty acids and then, consequently, gluconeogenesis and hyperglycaemia. Inhibitors of CPT have been described in J. Med. Chem., 1995, 38(18), p. 3448-50, and in the relevant European patent application EP-A-574355 as potential derivatives with hypoglycaemic action.

The international patent application WO99/59957 in the name of the Applicant describes and claims a class of derivatives of butyric acid which have displayed inhibitory action on CPT. An example of these compounds is R-4-trimethyl ammonium-3-(tetradecyl carbamoyl)-aminobutyrate (ST1326).

It has recently been demonstrated that the inhibition of CPT-1 in the hypothalamus, produced experimentally by administering intracerebroventricular inhibitors (icv), is capable of significantly and consistently reducing, in terms of extent and duration of the effect, food intake and gluconeogenesis (Nature Medicine, 2003, 9(6), 756-761). This property has also been demonstrated using the compound ST1326.

As regards the inhibition of CPT-1 it would therefore be important to be able to synthesize compounds which are able to cross the blood-brain barrier to be able to inhibit the CPT-1 in the hypothalamus and therefore have compounds which are effective in reducing food intake and gluconeogenesis. These compounds as drugs would therefore be beneficial in the treatment of obesity and/or diabetes.

### DESCRIPTION OF THE INVENTION

The present invention meets this requirement and, in particular, relates to new inhibitors of carnitine palmitoyl transferase with the following formula (I):

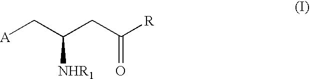

where:
A is selected among —N(R₂R₃), —N(R₂R₃R₄)^{⊕} and —C(R₂R₃R₄), in which the same or different R₂, R₃, R₄ are selected among H, alkyl C₁-C₂, phenyl, phenyl-alkyl C₁-C₂;
R is selected among —OH, —O^{⊖}, linear or branched alkoxy C₁-C₄, optionally replaced by a carboxy or alkoxycarbonyl group C₁-C₄, or the group Y-Z, in which:
Y=—O—(CH₂)ₙ—O—, —O—(CH₂)ₙ—NH—, —S—(CH₂)ₙ—O—, —S—(CH₂)ₙ—NH—, where n is selected among 1, 2 and 3, or —O—(CH₂)ₙ—NH—, where n is selected among 0, 1, 2 and 3; and

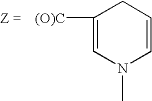

R₁ is selected among —COOR₅, —CONHR₅, —SOR₅, —SONHR₅, —SO₂R₅ and —SO₂NHR₅, in which
R₅ is a saturated or unsaturated, linear of branched alkyl C₁-C₂₀, replaced by aryl C₆-C₁₀, aryloxy C₆-C₁₀, heteroaryl C₄-C₁₀ containing 1 or more atoms selected among N, O and S, heteroaryloxy C₄-C₁₀ containing 1 or more atoms selected among N, O and S, in turn replaced by saturated or unsaturated, linear or branched alkyl or alkoxy C₁-C₂₀;
on condition that, when A is —N(R₂R₃R₄)^{⊕} and R₂, R₃ and R₄ are the same and are alkyls, R is different from —OH or —O^{⊖}.

As regards other compounds known to be structurally and functionally similar, the compounds of the present invention have the advantage of crossing the BBB more easily, at the same time maintaining excellent levels of inhibition of the activity of CPT. They are therefore able to inhibit the activity of CPT in the hypothalamus thus presenting the effects in the reduction in food intake, as described above.

Preferably R₁ is —CONHR₅ and R₅ is a linear or branched alkyl, saturated or unsaturated, containing between 7 and 20 carbon atoms. The preferred R₅ groups are therefore selected among heptyl, octyl, nonyl, decyl, undecyl, dodecyl, tridecyl, tetradecyl, pentadecyl, hexadecyl, heptadecyl, octadecyl, nonadecyl and eicosyl.

Preferably R₂ or R₃ or both are methyl.

Depending on the meanings of the radicals A, R₁, R₂, R₃, R₄, R₅, Y and Z, in the compounds of formula (I), one or more chiral centres (on carbon or nitrogen atoms) may be present. For the purposes of the present invention it is pointed out that each of the products of formula (I) can exist both as a racemic mixture RIS, and in the separate isomeric forms R and S.

The products of formula (I), in which A is —N(R₂R₃R₄)^{⊕} and R is different from —OH and —O^{⊖}, can exist only as salts with pharmacologically acceptable anions. These anions are here identified by the radical X⁻.

The products of formula (I) in which A is —N(R₂R₃) can exist as internal salts, as salts with pharmacologically acceptable acids and also in anionic form without a positive net charge on the nitrogen in group A.

The products of formula (I) in which A does not contain nitrogen can exist in neutral or anionic form.

The present invention covers all these different possibilities of salification for the compounds of formula (I).

Preferred pharmaceutically acceptable salts (I) are acid addition salts formed with pharmaceutically acceptable acids like hydrochloride, hydrobromide, hydroiodide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

Suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Sodium salts are particularly preferred.

The compounds of formula (I) which do not contain positive or negative net charges are expected to be very efficient in crossing the blood-brain barrier.

The following are preferred compounds of formula (I ):
(R)-4-(dimethyl amino)-3-(tetradecyl carbamoyl)-methyl aminobutyrate;
(R)-4-(dimethyl amino)-3-(tetradecyl carbamoyl)-aminobutyric acid;
(R)-4-(trimethyl amino)-3-(tetradecyl carbamoyl)-methyl aminobutyrate chloride;
(R)-4-trimethylammonium-3-(tetradecylcarbamoyl)-amino-butyrate of {2[-(N-methyl-(1,4-dihydro-pyridine)-3-yl)carbonyl]-amino}ethyl iodide; and
(R)-4-trimethylammonium-3-(tetradecylcarbamoyl)-amino-butyrate of -3-(methoxycarbonyl)-propyl bromide.

The synthesis and the structure of these compounds is reported in detail in the section entitled Examples.

The products of formula (I) can be prepared using reactions known in the state of the art.

Examples of these reactions are reported in WO99/59957, Eur. J. Org. Chem. 2003, 4501-4505, Eur. J. Med. Chem. 39 (2004), 715-727 and Helv. Chim. Acta 1996, 79, 1203-1216.

As an example of this process, FIG. 1 shows a synthetic Scheme for compounds of formula (I), in which A is —N(R₂R₃R₄)^{⊕}, R₁ has any of the indicated meanings, R₂, R₃ and R₄ are methyl and R has any of the indicated meanings. The following steps may be followed in this case.

### Step a

To compound 1 obtained as described in Eur. J. Org. Chem. 2003, 4501-4505 a solution of dimethylamine in CH₃OH or THF, preferably THF, is added. The reaction mixture is left under magnetic stirring for a time ranging from 4 to 8 hours, preferably 4 hours, at a temperature ranging from 20° C. to 40° C. preferably 25° C. The residue obtained by evaporation of the solvent is triturated several times with a polar solvent preferably diethyl ether. The ethereal layers are evaporated under vacuum and the residue purified by silica gel chromatography.

### Step b

The preparation of compound 3, is performed by reacting compound 2 with an inorganic acid in water such as hydrochloric acid or hydrogen bromide preferably HBr/H₂O 48% in presence of an aromatic alcohol preferably phenol for a time ranging from 24 to 48 hours at a temperature ranging from 130 to 140° C.

### Step c

Preparation of compound 4 is performed by reacting 3 with an alcohol preferably methanol and an acidic chloride such as oxalyl chloride or thionyl chloride, preferably thionyl chloride at a temperature ranging from 0 to 40° C., for a time ranging from 12 to 24 hours.

### Step d

Compound 5 (R=alkoxy) is obtained first by reacting 4 with an appropriate reacting product selected among alkylisocyanate, alkylchloroformates, alkylsulfonylchloride, preferably alkylisocyanatealkylisocianate in anhydrous a polar organic solvent such as CH₃OH or DMF or DMSO, preferably CH₃OH, in presence of an organic base, preferably triethylamine, in the ratio ranging from 1:2 to 1:5, preferably 1:3, for a time ranging from 24 to 48 hours at a temperature ranging from 20 to 30° C. The pure product is obtained by silica gel chromatography. Finally, compound 5 (R=OH) is obtained by acidic hydrolysis performed by inorganic acid, preferably hydrochloric acid, ranging from 1N to 6N, preferably 2N, at 25° C. for a time ranging from 3 to 7 days.

### Step h

Compound 4′ obtained as described in WO44/59957 (WO99/59957), is esterified by reacting with anhydrous alcohol such as CH₃OH, CH₃CH₂OH, isopropanol, preferably CH₃OH and an acidic chloride such as oxalyl chloride or thionyl chloride, preferably thionyl chloride or by bromoalkylmethoxycarbonile in anhydrous solvent as DMF, CH₃CN, preferably anhydrous DMF.

Pure compounds 5′ are obtained by solvent evaporation.

### Step e

Compound 6 is obtained by reaction of 4′ and hydroxyalkylnicotinamide with condensing agent as DCC or CDI, preferably DCC (ratio 1:1:4-5) in polar aprotic solvent such as CH₂Cl₂, CHCl₃ or CH₃CN, preferably CH₂Cl₂, for a time ranging from 24 to 36 hours at a ranging temperature from 20 to 30° C., preferably 25° C.

### Step f

Product 7 is obtained by methylation of 6 by methylating agent such as methyliodide in ratio 1:10-15 in anhydrous polar aprotic solvent such as CH₃CN, Et₂O or DMF, preferably anhydrous CH₃CN at ranging temperature from 20 to 30° C. for a ranging time from 24 to 36 hours.

### Step g

Product 8 is obtained by 7 by reaction with Na₂S₂O₄ (ratio 1:1-2), in presence of an inorganic base preferably NaHCO₃, using as solvent a mixture of CH₂Cl₂ or CHCl₃, preferably CH₂Cl₂ in water (9:2). PureFinal pure 8 is obtained by extraction with organic solvent as CH₂Cl₂ or CHCl₃ and evaporation.

The compounds of formula (I) have inhibitory activity on carnitine palmitoyl transferases. This action makes it possible to use them in the treatment and/or in the prevention of obesity, hyperglycaemia, diabetes and associated disorders such as, for example, diabetic retinopathy, diabetic neuropathy and cardiovascular disorders. The compounds of formula (I) are also used in the prevention and treatment of cardiac disorders such as congestive heart failure.

The inhibitory action of the compounds of formula (I) takes place mainly on isoform 1 of carnitine palmitoyl transferase (CPT-1) and, in particular, also in the hypothalamus.

A further object of the present invention are pharmaceutical compounds containing one or more of the products of formula (I) described earlier, in combination with excipients and/or pharmacologically acceptable diluents.

The compounds in question may, together with the compounds of formula (I), contain known active principles.

The pharmaceutical compositions according to the present invention may be adapted for oral, parenteral, rectal and transdermal administration. The oral forms include capsules, tablets, granules, powders, syrups and elixirs. The parenteral forms include solutions or emulsions.

The dosage of the products of the present invention vary depending on the type of product used, the route of administration and the degree of development of the disease to be treated. In general an effective therapeutic effect can be obtained at dosages between 0.1-100 mg/kg.

The invention also includes the use of the products of formula (I) for the preparation of drugs with hypoglycaemic and anti-obesity action.

A further embodiment of the invention is a process for the preparation of pharmaceutical compositions characterised by mixing one or more compounds of formula (I) with suitable excipients, stabilizers and/or pharmaceutically acceptable diluents.

Another object of the present invention is the method of treating a mammal suffering from hyperglycaemia, diabetes, obesity and associated disorders as reported before, comprising administering a therapeutically effective amount of the compound of formula (I).

The present invention is now illustrated by the following non-limitative examples.

### DESCRIPTION OF THE DRAWINGS

FIG. 1 shows a synthetic Scheme for compounds of formula (I), in which A is —N(R₂R₃R₄)^{⊕}, R₁ has any of the meanings indicated for formula (I) compounds, R₂, R₃ and R₄ are methyl and R has any of the meanings indicated for formula (I) compounds.

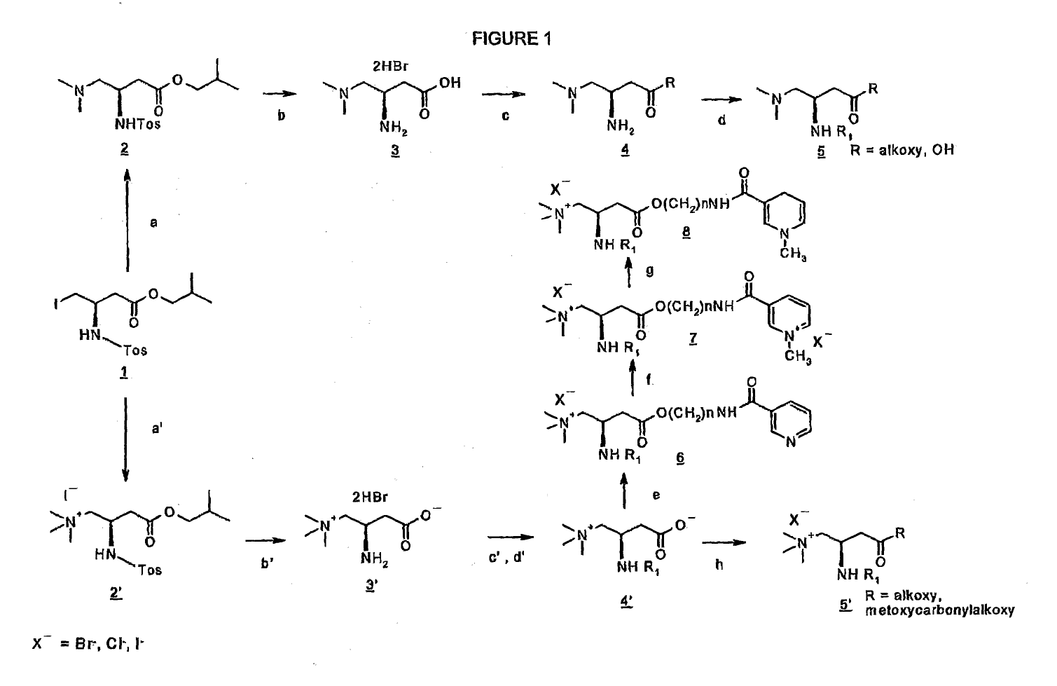

### EXAMPLES

### Preparation of the Compounds of Formula (I)

### Example 1

### Preparation of methyl (R)-4-(dimethylamino)-3-(tetradecylcarbamoyl)-amino-butyrate (ST 2669)

Preparation of the Intermediate Isobutyl (R)-4-Dimethylamino-3-(toluene-4-sulfonyl amino)-butyrate

To 10 g (22.76 mmol) of (R)-4-iodo-3-(toluene-4-sulfonyl amino) butyrate of isobutyl (preparation as described in Eur. J. Org. Chem. 2003, 4501-4505) dimethyl amine (2.0 M in THF) (28.5 ml, 57 mmol) was added. The suspension thus obtained was left under magnetic agitation for 4 hours. After this time the solvent was evaporated under vacuum and the residue was triturated several times with ethyl ether. The combined ether phases were evaporated under vacuum and the residue obtained was purified by means of chromatography on a silica gel column using as the eluent CHCl₃/MeOH 99.5:0.5 to give 5.84 g of the desired product (72% yield). TLC: silica gel, eluent CHCl₃/MeOH 9.6:0.4, R_{f}=0.33; ¹H NMR (300 MHz, MeOH-d₄) δ: 7.84 (d, 2 H, ArH), 7.46 (d, 2 H, ArH), 3.88-3.81 (m, 2H, CH₂), 3.80-3.63 (m, 1H, CH), 2.61-2.44 (m, 5H, CH, CH₃), 2.38-2.25 (m, 2H, CH₂), 2.15 (s, 6H, CH₃), 2.01-1.87 (m, 1H, CH), 1.00 (d, 6H, CH₂); HPLC: SCX column (5 μm-4.6×250 mm), mobile phase CH₃CN/50 mM NH₄H₂PO₄ 60/40 v/v, room temperature, flow rate: 0.8 ml/min, detector: UV 205 nm, retention time: 6.73 min.

### Preparation of the Intermediate Methyl (R)-3-amino-4-(dimethylamino)-butyrate Dibromohydrate

To the mixture of the above prepared compound (3.0 g, 8.4 mmol) and phenol (2.37 g, 25.2 mmol) HBr 48% in H₂O (45 ml) was added. The obtained solution was brought to 135° C. for one night (N.B. the oil bath must already be up to temperature when the flask containing the solution is introduced). After this time the solution was diluted with water and extracted twice with AcOEt and the aqueous phase was evaporated under vacuum. The residue obtained was dissolved in acetonitrile and evaporated under vacuum several times. (R)-3-amino-4-(dimethyl amino) butyric dibromohydrate (2.47 g) was obtained (¹H NMR:(300 MHz, MeOH-d₄) δ: 3.60 (m, 1 H, CH), 2.70-2.42 (m, 4H, 2CH₂), 2.40 (s, 6H, 2 CH₃)), which was used as such in the following reaction.

To a solution of the acid prepared as described above (2.47 g, 8 mmol) in anhydrous methanol (7.5 ml), cooled to 0° C., thionyl chloride (2.78 g, 1.7 ml, 24 mmol) was added. The reaction mixture was left under magnetic stirring for ten minutes at 0° C., then, for the same period at room temperature and finally for 12 hours at 40° C. After this time the reaction mixture was dried under vacuum and purified by means of flash chromatography on silica gel using as the eluent a gradient from CHCl₃/MeOH 9:1 to CHCl₃/MeOH 7:3. The intermediate dibromohydrate (1.37 g, 71% yield) was obtained [α]²⁰D=−15.1° (c=1.6%, MeOH); ¹H NMR (300 MHz, MeOH-d₄) δ: 3.80 (s, 3H, CH₃), 3.63-3.57 (m, 1H, CH), 2.78-2.35 (m+s, 10H, CH₂, CH₃); A.E. in conformity with C₇H₁₈Br₂N₂O₂.

### Preparation of Methyl (R)-4-(dimethylamino)-3-(3-tetradecyicarbamoyl)-amino-butyrate (ST2669)

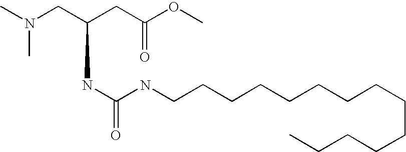

To a solution of methyl (3R)-3-amino-4-(dimethylamino)-butyrate dibromohydrate (1.28 g, 3.97 mmol) in anhydrous methanol (50 ml) triethyl amine (1.20 g, 1.65 ml, 11.91 mmol) was first added, followed by tetradecyl isocyanate (1.42 g, 1.63 ml, 5.95 mmol) at 0° C. The reaction mixture was left under magnetic stirring for 24 hours at room temperature, then the solvent was evaporated under vacuum. The crude product obtained was dissolved in EtOAc and washed with H₂O then with saturated solution of Na₂CO₃. The organic phase was evaporated under vacuum and the residue is purified by means of flash chromatography on silica gel eluting with CHCl₃/MeOH 9.6/0.4. The desired product (1.22 g, 77% yield) was obtained. M.p. 44-45° C.; TLC: silica gel, eluent CHCl₃/MeOH 8:2, R_{f}=0.32; [α]²⁰D=−28.4° (c=1%, MeOH); ¹H NMR (300 MHz, MeOH-d₄) δ: 4.21-4.10 (m, 1H, CH), 3.65 (s, 3H, CH₃), 3.08 (t, 2H, CH₂), 2.59-2.45 (m, 2H, CH₂), 2.43-2.29 (m, 2H, CH₂), 2.24 (s, 6H, CH₃), 1.45 (m, 2H, CH₂), 1.28 (s, 22H, CH₂), 0.89 (t, 3H, CH₃); HPLC: SCX column (5 μm-4.6×250 mm), mobile phase: CH₃CN/50 mM NH₄H₂PO₄ 60/40 v/v, room temperature, flow rate: 0.8 ml/min, detector: UV 205 nm, retention time: 5.69 min; MS (ESI) 400 [M+1]⁺, 422 [M+Na]⁺; H₂O; A.E. in conformity with C₂₂H₄₅N₃O₃.

### Example 2

### Preparation of (R)-4-(dimethylamino)-3-(tetradecylcarbamoyl)-amino-butyric Acid (ST2837)

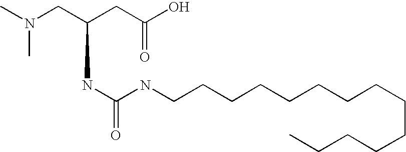

To the product prepared as described in example 1 (ST2669, 0.180 g, 0.45 mmol) an aqueous solution of HCl 6N (3.5 ml) was added. The reaction mixture was left under magnetic stirring at room temperature for one week. After this time the reaction mixture was evaporated under vacuum and the residue was purified by means of flash chromatography on silica gel using as the eluent a gradient from CHCl₃/MeOH 8:2 to CHCl₃/MeOH 1:1. The desired product (67 mg, 38% yield) was obtained. TLC: silica gel, eluent: CHCl₃/MeOH 7:3, R_{f}=0.40; [α]²⁰D=−8.4° (c=0.5%, MeOH); ¹H NMR (300 MHz, MeOH-d₄) δ: 4.34-4.26 (m, 1H, CH), 3.30-3.10 (m, 4H, CH₂), 2.90 (s, 6H, CH₃), 2.58 (d, 2H, CH₂), 1.55 (m, 2H, CH₂), 1.40 (s, 22H, CH₂), 1.00 (t, 3H, CH₃); HPLC: SCX column (5 μm-4.6×250 mm), mobile phase: CH₃CN/50 mM NH₄H₂PO₄ 40/60 v/v, pH=3.7 (H₃PO₄), room temperature, flow rate: 0.8 ml/min, detector: UV 205 nm, retention time: 8.09 min; K.F.=2.3% H₂O; A.E. in conformity with C₂₁H₄₃N₃O₃.

### Example 3

### Preparation of Methyl (R)-4-trimethylammonium-3-(tetradecylcarbamoyl)-amino-butyrate Chloride (ST2822)

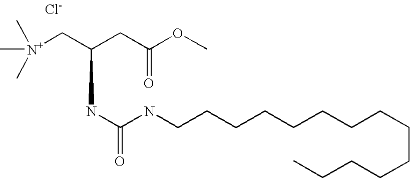

To the solution of (R)-4-trimethylammonium-3-(tetradecylcarbamoyl)-amino-butyrate (ST1326, prepared as described in WO99/59957) (1.20 g, 3.00 mmol) in anhydrous MeOH (6 ml) thionyl chloride (1.80 g, 1.10 ml, 15.13 mmol) was added added, at 0° C. and drop by drop, leaving the solution under stirring at 40° C. for 72 hours. After drying under vacuum, the reaction mixture was washed with anhydrous ethyl ether. The oil obtained was purified using flash chromatography on silica gel (eluent used MeOH/CHCl₃ 1:1). The product obtained was dissolved in anhydrous dichloromethane and filtered through a Millex-HV Hydrophilic PVDF 0.45 μm (Millipore) filter. By evaporating the solvent under vacuum the desired product was obtained (164 mg, 12% yield). TLC: silica gel, eluent (42:7:28:10.5:10.5 CHCl₃/isopropanol/MeOH/CH₃COOH/H₂O), R_{f}=0.83; [α]²⁰D=−8.5° (c=1%, MeOH); ¹H NMR (300 MHz, MeOH-d₄) δ: 4.65 (br s, 1H, CH), 3.70 (s, 3H, CH₃), 3.65-3.40 (m, 2H, CH₂); 3.20 (s, 9H, CH₃), 3.10 (t, 2H, CH₂), 2.70 (m, 2H, CH₂), 1.45-1.40 (m, 2H, CH₂), 1.30 (s, 22H, CH₂), 0.90 (t, 3H, CH₃); HPLC: SCX column (5 μm-4.6×250 mm), mobile phase CH₃CN/50 mM NH₄H₂PO₄ 40/60 v/v, room temperature, flow rate: 0.8 ml/min, detector: UV 205 nm, retention time: 10.94 min; MS (ESI) 355 [M-(CH₃)₃N]⁺, 414 [M]⁺; K.F.=1.8% H₂O; A.E. in conformity with C₂₃H₄₈N₃O₃Cl.

### Example 4

### Preparation of (R)-4-trimethylammonium-3-(tetradecylcarbamoyl)-amino-butyrate of {2[-(N-methyl-(1,4-dihydro-pyridine)-3-yl)carbonyl]-amino}ethyl Iodide (ST3496)

### Preparation of the Intermediate N-(2-hydroxy-ethyl)-nicotinamide

SOCl₂ (455 μl, 6.26 mmol) ) was added to a suspension of nicotinic acid (0.385 g, 3.13 mmol) in anhydrous toluene (15 ml) and the reaction mixture was refluxed at 140° C. for 4 hours. Then the clear solution was cooled and the solvent was removed under vacuum. The solid residue was washed three times with diethyl ether and fresh anhydrous toluene (15 ml) and ethanolamine (756 μl, 12.52 mmol) were added. The mixture was warmed up to 50° C. overnight.

Then the solvent was removed under vacuum and the solid residue was purified by silica gel chromatography using as eluent dichloromethane/methanol 9.2/0.8. The desired product was obtained as a white solid (450 mg, 86% yield). m.p.=84.5-85.5° C.; ¹H NMR (300 MHz, DMSO-d₆) δ: 9.00 (s, 1H, NH), 8.68, (m, 2H, Ar), 8.17 (d, 1H, Ar), 7.60 (m, 1H, Ar), 4.74 (m, 1H, OH), 3.51 (m, 2H, CH₂), 3.36 (m, 2H, CH₂).

### Preparation of the Intermediate (R)-4-trimethylammonium-3-(tetradecylcarbamoyl)-amino-butyrate of {2-[(pyridin-3-yl)carbonyl]-amino}ethyl Chloride

To a solution of N-(2-hydroxy-ethyl)-nicotinamide (0.274 g, 1.65 mmol) in anhydrous dichloromethane (16 ml) (R)-4-trimethylammonium-3-(tetradecylcarbamoyl)-amino-butyrate hydrochloride (0.719 g, 1.65 mmol, prepared by adding an equivalent of hydrochloric acid 1N to ST1326 prepared as described in WO99/59957) and dicyclohexylcarbodiimide (DCC) (1.018 g, 5.00 mmol) were added. The reaction mixture was left overnight at room temperature under magnetic stirring. Then the mixture was filtered and the organic layer was concentrated under vacuum. The residue was washed several times with diethyl ether to give, after desiccation under vacuum, the desired product as a white solid (769 mg, 79% yield). TLC: silica gel, eluent CHCl₃/isopropanol/MeOH/CH₃COOH/H₂O 42:7:28:10.5:10.5, Rf=0.5; ¹H NMR (300 MHz, MeOH-d₄) δ: 9.05 (d, 1H, Ar), 8.70 (d, 1H, Ar), 8.30 (dm, 1H, Ar), 7.55 (m, 1H, Ar), 4.70 (brs, 1H, CH), 4.31 (t, 2H, CH₂), 3.70 (t, 2H, CH₂), 3.70-3.50 (m, 2H, CH₂), 3.25 (s, 9H, N(CH₃)₃), 3.04 (t, 2H, CH₂), 2.68 (t, 2H, CH₂), 2.43 (brm, 2H, CH₂), 2.28 (s, 24H, (CH₂)₁₂), 0.95 (t, 3H, CH₃); MS (ESI) 548 [M]⁺.

### Preparation of the Intermediate (R)-4-trimethylammonium-3-(tetradecylcarbamoyl)-amino-butyrate of {2[-(N-methylpyridin-3-yl)carbonyl]-amino}ethyl Diiodide (ST3474)

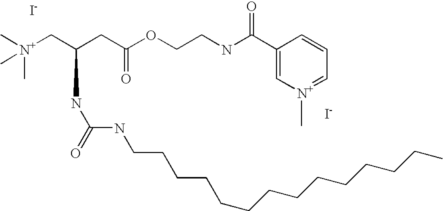

Methyl iodide (747 μl, 12.00 mmol) was added to a solution of (R)-4-trimethylammonium-3-(tetradecylcarbamoyl)-amino-butyrate {[(pyridin-3-yl)carbonyl]-amino}ethyl chloride (0.700 g, 1.2 mmol) in anhydrous CH₃CN (40 ml) and the so obtained reaction mixture was left under magnetic stirring at room temperature overnight. Then the solvent was removed under vacuum and the desired product (957 mg, 98% yield) was obtained. M.p.: 179-181° C.; TLC: silica gel, eluent CHCl₃/isopropanol/MeOH/CH₃COOH/H₂O 42:7:28:10.5:10.5, Rf: 0.3; [α]²⁰D=−0.80 (c=2%, MeOH); ¹H NMR (300 MHz, MeOH-d₄) δ: 9.48 (s, 1H, Ar), 9.00 (dd, 2H, Ar), 8.20 (t, 1H, Ar), 4.75 (brm, 1H, CH), 4.51 (s, 3H, CH₃), 4.32 (t, 2H, CH₂), 3.70 (m, 4H, 2CH₂), 3.25 (s, 9H, N(CH₃)₃), 3.10 (t, 2H, CH₂), 2.75 (dd, 2H, CH₂), 1.42 (brm, 2H, CH₂), 1.30 (s, 22H, (CH₂)₁₁), 0.90 (t, 3H, CH₃); HPLC: Column: Waters Spherisorb S5 SCX(4.6×250 mm), mobile phase: CH₃CN/NH₄H₂PO₄ 200 mM, 60/40 v/v, pH as it is, room temperature, flow rate: 1.0 ml/min, detector: UV 254 nm, retention time: 20.60 min; MS (ESI) 281 [M]⁺/2; K.F.=2.70% H₂O; A.E. in conformity with C₃₁ H₅₇N₅O₄I₂.

### Preparation of (R)-4-trimethylammonium-3-(tetradecylcarbamoyl)-amino-butyrate of {2[-(N-methyl-(1,4-dihydro-pyridine)-3-yl)carbonyl]-amino}ethyl Iodide (ST3496)

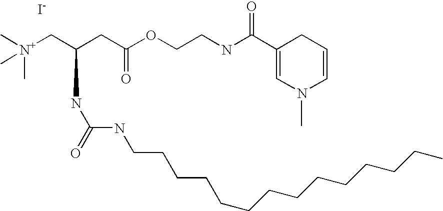

To a solution of (R)-4-trimethylammonium-3-(tetradecylcarbamoyl)-amino-butyrate of {2[-(N-methylpyridin-3-yl)carbonyl]-amino}ethyl diiodide, prepared as above described (ST3474, 0.100 g, 0.12 mmol) in degased water (18 ml) chilled to 0° C. and under argon atmosphere NaHCO₃ (0.200 g, 1.2 mmol), Na₂S₂O₄ (0.046 g, 0.26 mmol), both dissolved in 11 ml of a mixture of water and dichloromethane 9/2 were added. The reaction mixture was left under magnetic stirring at 0° C. for 15 minutes and then for other 30 minutes at room temperature. The organic layer was then separated from water and the aqueous layer was extracted several times with dichloromethane. The combined organic layers were dried over Na₂SO₄ then concentrated to give the final product (0.084 g, 94% yield), which was kept under vacuum to avoid degradation. TLC: silica gel, eluent CHCl₃/isopropanol/MeOH/CH₃COOH/H₂O 42:7:28:10.5:10.5, Rf: 0.7; ¹H NMR (300 MHz, DMSO-d₆) δ: 7.16 (t, 1H, NH), 6.80 (s, 1H, CH═CH), 6.25 (m, 2H, 2NH), 5.80 (d, 1H, CH═CH), 4.60 (m, 1H, CH═CH), 4.48 (brm, 1H, CH), 4.05 (m, 2H, CH₂), 3.75-3.05 (brm, 4H, 2CH₂), 3.09 (s, 9H, N(CH₃)₃), 2.95 (brs, 4H, 2CH₂), 2.87 (s, 3H, NCH₃), 2.57 (brt, 2H, CH₂), 1.32 (brs, 2H, CH₂), 1.20 (s, 22H, (CH₂)₁₁), 0.82 (t, 3H, CH₃); MS (ESI) 564 [M]⁺; A.E. in conformity with C₃₁ H₅₈N₅O₄I.

### Example 5

### Preparation of (R)-4-trimethylammonium-3-(tetradecylcarbamoyl)-amino-butyrate of -3-(methoxycarbonyl)-propyl Bromide (ST3193)

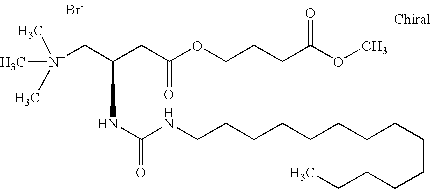

Methyl-4-bromo-butyrate was added (0.460 mg 2.54 mmol) to a solution of (R)-4-trimethylammonium-3-(tetradecylcarbamoyl)-amino-butyrate (1.015 g, 2.54 mmol) in 12 ml of anhydrous DMF. The reaction mixture was kept at 50° C. under magnetic stirring overnight. The solvent was then evaporated to give the desired product as a pale yellow waxy solid (1.108 g, 87% yield).; TLC: silica gel, eluent 42:7:28:10.5:10.5 CHCl₃/isopropanol/MeOH/CH₃COOH/H₂O, R_{f}=0.6; [α]²⁰D=−7.60 (c=1%, MeOH); ¹H NMR (300 MHz, MeOH-d₄) δ: 4.67 (brm, 1H, CH), 4.17 (t, 2H, CH₂), 3.70 (s, 3H, CH₃), 3.72-3.46 (m, 2H, CH₂), 3.30 (s, 9H, CH₃), 3.12 (t, 2H, CH₂), 2.68 (m, 2H, CH₂), 2.44 (t, 2H, CH₂), 1.96 (brm, 2H, CH₂), 1.48 (brs, 2H, CH₂), 1.30 (s, 24H, (CH₂)₁₂), 0.91 (t, 3H, CH₃); HPLC: SCX column (5 μm-4.6×250 mm), mobile phase: CH₃CN/NH₄H₂PO₄ 50 mM, 40/60 v/v, pH 3.6, room temperature, flow rate: 0.8 ml/min, detector: UV 205 nm, retention time: 10.08 min; MS (ESI) 500 [M]⁺; K.F.=0.88% H₂O; A.E. in conformity with C₂₇H₅₄N₃O₅Br.

### Determination of the Pharmacological Activity of the Compounds of Formula (I)

### Test 1: Determination of the Inhibitory Action of CPT

The inhibition of CPT was evaluated on fresh mitochondrial preparations obtained from the liver or heart of Fischer rats, fed normally; the mitochondria taken from the liver or heart are suspended in a 75 mM sucrose buffer, EGTA 1 mM, pH 7.5. 100 μl of a mitochondrial suspension, containing 50 μM of [¹⁴C] palmitoyl-CoA (spec.act. 10000 dpm/mole) and 10 mM of L-carnitine, are incubated at 37° C. in the presence of stepped concentrations (0-3 mM) of product under examination.

Reaction time: 1 minute.

The IC₅₀ is then determined. The results are reported in Table 1.

TABLE 1
IC₅₀ of the inhibition curve of CPT1 in rat mitochondria

| Substance | IC₅₀ of CPT1 liver (μM) | IC₅₀ of CPT1 heart (μM) |
|---|---|---|
| ST1326 | 0.36 | 48.8 |
| ST2837 | 5.7 | 70 |

### Test 2: Determination of the Production of β-hydroxybutyrate Stimulated by Oleate

The synthesis of β-hydroxybutyrate is an indication of the activity of CPT. In fact the production of ketone bodies, end-products of mitochondrial beta-oxidation, is linked to the activity of CPT.

Hepathocytes preparations obtained according to the technique described in Venerando R. et al. (1994) Am. J. Physiol. 266: C455-C461] are used. The hepatocytes are incubated at 37° C. in KRB bicarbonate buffer at pH 7.4, glucose 6 mM, 1% BSA in a O₂/CO₂ 95/5% atmosphere at the concentration of 2.5×10⁶ cells/ml. After a preincubation period of 40 min. with a compound to be assayed at different concentrations, the first series of samples is taken (T_{o min}) and the oleate is added (1 mM final in KRB+BSA 1.4%). After 20 mins the second sample is taken (T_{20 min}).

### Test 3: β-hydroxy Butyrate in the Serum of Treated Rats

Fischer rats, normally fed, are kept in a fasting state for 24 hours and then treated with the compounds to be tested. One hour after the treatment the animals are sacrificed and the serum concentrations of β-hydroxy butyrate are determined.

### Other Tests

The ability of these compounds to cross the blood-brain barrier in rats or mice after oral or intravenous administration is measured on brain homogenates using HPLC-MS techniques. From preliminary data the compounds of the invention were shown to be able to efficiently cross the bolld-barrier.

Furthermore the evaluation of food intake after oral or intravenous administration is determined in rats with access to food ad libitum or on a time-restricted basis, for acute or fasting administration.

Finally the lowering of glycaemia for oral or intracerebroventricular administration in diabetic mice, for example db/db mice, is measured.

1. Compound in the racemic form (R,S) or in their R and S enanthiomeric forms, and their pharmacologically acceptable salts, having the structure of formula (I): 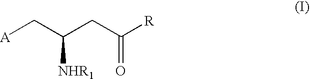
where:
A is selected among —N(R₂R₃), —N(R₂R₃R₄)^{⊕} and —C(R₂R₃R₄), in which the same or different R₂, R₃, R₄ are selected among H, alkyl C₁-C₂, phenyl, phenyl-alkyl C₁-C₂;
R is selected among —OH, —O^{⊖}, linear or branched alkoxy C₁-C₄, optionally replaced by a carboxy or alkoxy carbonyl group C₁-C₄, or the group Y-Z, in which:
Y=—O—(CH₂)ₙ—O—, —O—(CH₂)ₙ—NH—, —S—(CH₂)ₙ—O—, —S—(CH₂)ₙ—NH—, where n is selected among 1, 2 and 3, or —O—(CH₂)ₙ—NH—, where n is selected among 0, 1, 2 and 3; and 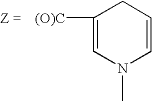
R₁ is selected among —COOR₅, —CONHR₅, —SOR₅, —SONHR₅, —SO₂R₅ and —SO₂NHR₅, in which
R₅ is a saturated or unsaturated, linear or branched alkyl C₁-C₂₀, replaced by aryl C₆-C₁₀, aryloxy C₆-C₁₀, heteroaryl C₄-C₁₀ containing 1 or more atoms selected among N, O and S, heteroaryloxy C₄-C₁₀ containing 1 or more atoms selected among N, O and S, in turn replaced by saturated or unsaturated, linear or branched alkyl or alkoxy C₁-C₂₀;
with the proviso that when A is —N(R₂R₃R₄)^{⊕} and R₂, R₃ and R₄ are the same and are alkyl, R is different from —OH or —O^{⊖}.

2. The compound according to claim 1, where R₂, R₃ and R₄ are methyl.

3. The compound according to claim 1, where R₁ is —CONHR₅.

4. The compound according to claim 3, where R₅ is a linear or branched, saturated or unsaturated alkyl containing from 7 to 20 carbon atoms.

5. The compound according to claim 4, where R₅ is selected among heptyl, octyl, nonyl, decyl, undecyl, dodecyl, tridecyl, tetradecyl, pentadecyl, hexadecyl, heptadecyl, octadecyl, nonadecyl and eicosyl.

6. The compound according to claim 1, which is (R)-4-(dimethyl amino)-3-(tetradecyl carbamoyl)-methyl aminobutyrate.

7. The compound according to claim 1 which is (R)-4-(dimethyl amino)-3-(tetradecyl carbamoyl)-aminobutyric acid.

8. The compound according to claim 1, which is (R)-4-(trimethyl amino)-3-(tetradecyl carbamoyl)-methyl aminobutyrate chloride.

9. The compound according to claim 1, which is (R)-4-trimethylammonium-3-(tetradecylcarbamoyl)-amino-butyrate of {2[-N-methyl-(1,4-dihydro-pyridine)-3-yl)carbonyl]-amino}ethyl iodide.

10. The compound according to claim 1, which is (R)-4-trimethylammonium-3-(tetradecylcarbamoyl)-amino-butyrate of -3-(methoxycarbonyl)-propyl bromide.

11. Process for the preparation of a compound of claim 1.

12. (canceled)

13. Pharmaceutical composition containing as active ingredient a compound according to claim 1 in combination with excipients and/or pharmaceutically acceptable diluents.

14. Process for the preparation of the pharmaceutical composition according to claim 13, comprising mixing a compound according to claim 1 with excipients, stabilizers and/or pharmaceutically acceptable diluents.

15. A method of treating disorders associated with hyperactivity of carnitine palmitoyl transferase, comprising administering an effective amount of a compound of claim to a mammal in need thereof.

16. The method according to claim 15, wherein the disorders are selected from the group consisting of the prevention and treatment of obesity, hyperglycaemia, diabetes and related disorders, and congestive heart failure.

17. Method of treating a mammal suffering from a condition selected from the group consisting of hyperglycaemia, diabetes, obesity and associated disorders, comprising administering a therapeutically effective amount of a compound according to claim 1.
